(19) United States
(12) Patent Application Publication
(10) Pub. No.: US 2025/0173870 A1
(43) Pub. Date: May 29, 2025

(54) SUBSTANCE INFORMATION IMAGE PROCESSING DEVICE, IMAGE PROCESSING METHOD, NON-TRANSITORY STORAGE MEDIUM, AND IMAGE PROCESSING SYSTEM

(71) Applicant: CANON KABUSHIKI KAISHA, Tokyo (JP)

(72) Inventor: TORU KIKUCHI, Tokyo (JP)

(21) Appl. No.: 18/956,669

(22) Filed: Nov. 22, 2024

(30) Foreign Application Priority Data
Nov. 27, 2023 (JP) 2023-200148

(51) Int. Cl. G06T 7/00

(52) U.S. Cl. CPC G06T 7/0014; G06T 2207/10081; G06T 2207/20081; G06T 2207/30004; G06T 2211/408

(57) ABSTRACT

An image processing device includes at least one memory storing instructions and at least one processor that, upon execution of the instructions, configures the at least one processor to acquire a first trained model trained using a training data set including, as training data, first CT image data based on first detection data that is captured by a first X-ray CT device and first substance information based on the first detection data, acquire second CT image data based on second detection data that is captured by a second X-ray CT device including an energy-integrating radiation detector using a detection method different from a detection method used by the first X-ray CT device, and infer second substance information from the second CT image data by using the first trained model.

### BACKGROUND

### Field

The present disclosure relates to an image processing device, an image processing method, and a storage medium that enable acquisition of highly accurate substance information from CT image data acquired by using an energy-integrating radiation detector.

### Description of the Related Art

It is known that photon counting X-ray CT devices (PCCT: photon counting computed tomography) and dual energy X-ray CT devices (DECT: dual energy computed tomography) can detect the amount of X-ray transmission in different X-ray energy ranges, and therefore, substances having different X-ray attenuation coefficients in different X-ray energy ranges can be discriminated using such characteristics (refer to, for example, Japanese Patent Laid-Open No. 2022-13679). For example, detection of abnormalities in blood vessels can be facilitated by visualizing substance information in a differentiated form between an iodine contrast agent for angiography and calcification.

### SUMMARY

In clinical practice, X-ray CT devices including energy-integrating X-ray detectors that are neither PCCT nor DECT are used. However, it is difficult to acquire discrimination information from detection data output from existing X-ray CT devices including energy-integrating radiation detectors.

The present disclosure provides an image processing device, an image processing method, a storage medium, and an image processing system that enable acquisition of highly accurate substance information even from CT image data acquired from an existing X-ray CT device including an energy-integrating X-ray detector.

According to an embodiment of the present disclosure, an image processing device includes at least one memory storing instructions and at least one processor that, upon execution of the instructions, configures the at least one processor to acquire a first trained model trained using a training data set including, as training data, first CT image data based on first detection data that is captured by a first X-ray CT device and first substance information based on the first detection data, acquire second CT image data based on second detection data that is captured by a second X-ray CT device including an energy-integrating radiation detector using a detection method different from a detection method used by the first X-ray CT device, and infer second substance information from the second CT image data by using the first trained model.

Further features of the present disclosure will become apparent from the following description of exemplary embodiments with reference to the attached drawings.

### BRIEF DESCRIPTION OF THE DRAWINGS

FIG. 1 is a block diagram of an example of an image processing system according to first to third embodiments.

FIG. 2 is a block diagram of an example of the hardware configuration of an image processing device according to the first to third embodiments.

FIG. 3 is a block diagram of an example of the functional configuration of the image processing device according to the first embodiment.

FIG. 4 is a block diagram of an example of the functional configuration of the image processing device according to the first embodiment.

FIG. 5 is a flowchart of an example of a first learning process according to the first embodiment.

FIG. 6 is a block diagram of an example of the functional configuration of the image processing device according to the first embodiment.

FIG. 7 is a flowchart of an example of an image display process according to the first embodiment.

FIGS. 8A and 8B illustrate examples of a display screen of the image processing device according to the first to third embodiments.

FIG. 9 is a block diagram of an example of the functional configuration of the image processing device according to the second embodiment.

FIG. 10 is a flowchart of an example of a second learning process according to the second embodiment.

FIG. 11 is a block diagram of an example of the functional configuration of the image processing device according to the third embodiment.

### DESCRIPTION OF THE EMBODIMENTS

The present disclosure is described in detail below with reference to exemplary embodiments and the accompanying drawings. The same reference numerals are used throughout the exemplary embodiments to designate the same or similar items, and description of the item is omitted as appropriate. The configuration described in the following embodiments is only an example, and the present disclosure is not limited to the illustrated configuration.

### First Embodiment

### System Configuration

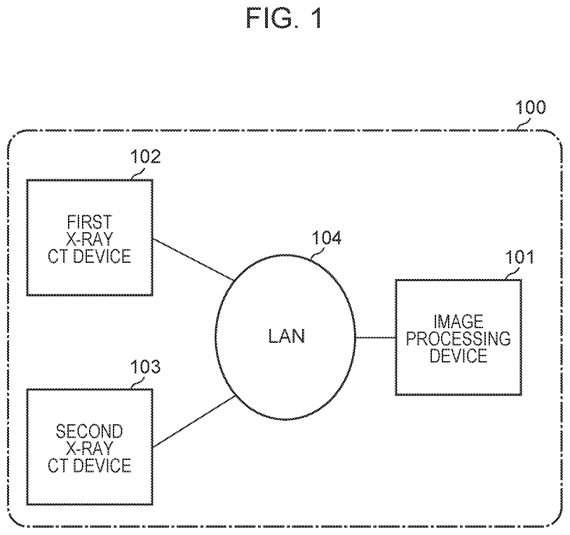

FIG. 1 is a block diagram of an example of an image processing system 100 according to the present embodiment. The image processing system 100 according to the present embodiment illustrated in FIG. 1 includes an image processing device 101, a first X-ray CT device 102, a second X-ray CT device 103, and a local area network (LAN) 104 that connects these devices with one another. According to the present embodiment, the first X-ray CT device 102 is a photon counting X-ray CT device. The second X-ray CT device 103 is an X-ray CT device using a detection method different from that in the first X-ray CT device 102 and, more specifically, an existing X-ray CT device including an energy-integrating X-ray detector. The image processing device 101 uses a training data set that includes training data including first CT image data and first substance information based on first detection data acquired from the first X-ray CT device 102. The image processing device 101 uses a trained model trained by using the training data set and generates substance information from second CT image data reconstructed on the basis of the detection data captured by the second X-ray CT device 103. As used herein, the term “substance information” refers to, for example, substance discrimination information. Examples of substance discrimination information includes region information in an image corresponding to a predetermined substance, information as to whether a substance in an image is present, and likelihood information of a predetermined region or pixels in the image being a predetermined substance.

The image processing device 101 may have a function of an image viewer that superimposes the generated substance information on the image data. The LAN 104 is a network including communication devices that follow a standard, such as IEEE (Institute of Electrical and Electronics Engineers) 802.3ab.

The configuration of the image processing system 100 is not limited thereto, and the image processing device 101 may be connected to a storage device, such as a database, that stores CT image data captured by the first X-ray CT device 102, the second X-ray CT device 103, or the like. The image processing system 100 may be configured to include the image processing device 101 and a storage device, such as a database, that stores the CT image data.

### Hardware Configuration

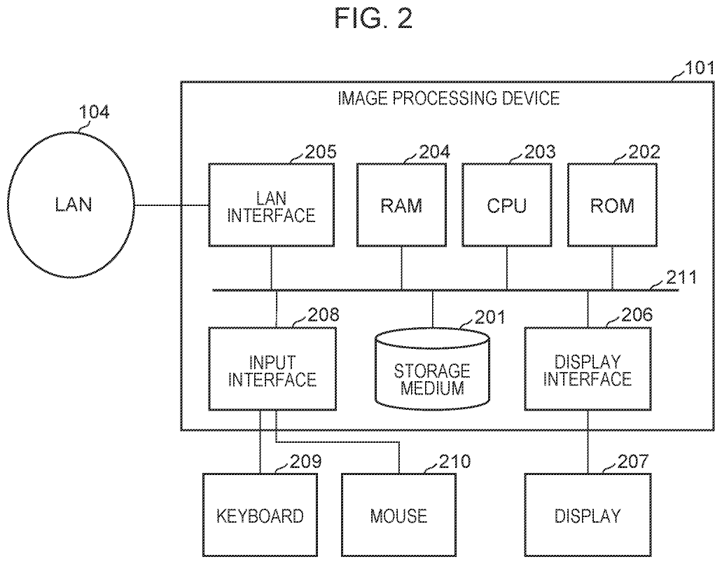

FIG. 2 is a block diagram of an example of the hardware configuration of the image processing device 101 according to the present embodiment. The image processing device 101 illustrated in FIG. 2 includes a storage medium 201, a read only memory (ROM) 202, a central processing unit (CPU) 203, and a random-access memory (RAM) 204. The image processing device 101 further includes a LAN interface 205, an input interface 208, a display interface 206, and an internal bus 211. A keyboard 209 and a mouse 210 are connected to the image processing device 101 via the input interface 208. A display 207 is also connected to the image processing device 101 via the display interface 206.

The storage medium 201 is a storage medium, such as a solid-state drive (SSD), that stores an operating system (OS), processing programs for performing a variety of processes according to the present embodiment, and a variety of information according to the present embodiment. The ROM 202 stores programs, such as a basic input output system (BIOS) for initializing the hardware, reading the OS stored in the storage medium 201, and starting the OS. The CPU 203 performs arithmetic processing when executing the BIOS, OS, and processing programs. The RAM 204 temporarily stores the processing program and a variety of data when the CPU 203 executes the BIOS, OS, and processing programs. The LAN interface 205 is an interface that supports the standard, such as IEEE802.3ab, and communicates via the LAN 104. The display 207 is a display device, such as a liquid crystal display (LCD), that displays a user interface screen. The display interface 206 converts screen information to be displayed on the display 207 into a display control signal and outputs the display control signal to the display 207. The input interface 208 receives a signal based on key pressing, button click, movement of coordinates, or the like from the keyboard 209 and the mouse 210. The internal bus 211 transmits signals when communication is performed between the blocks.

### Functional Configuration

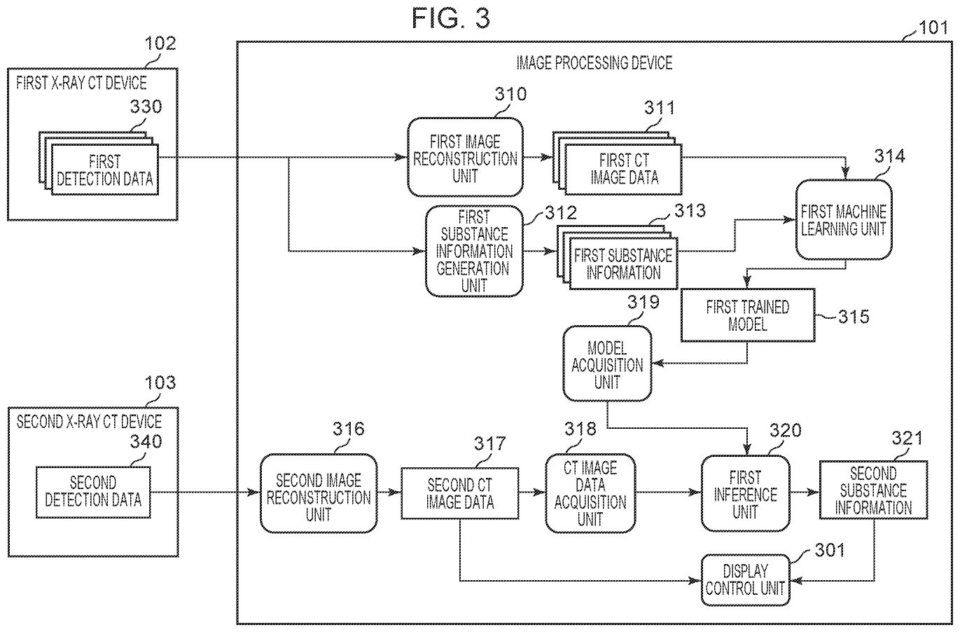

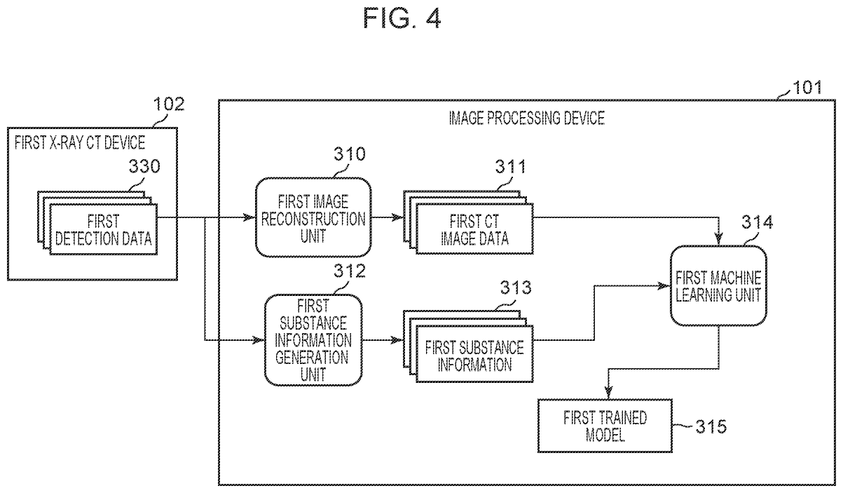

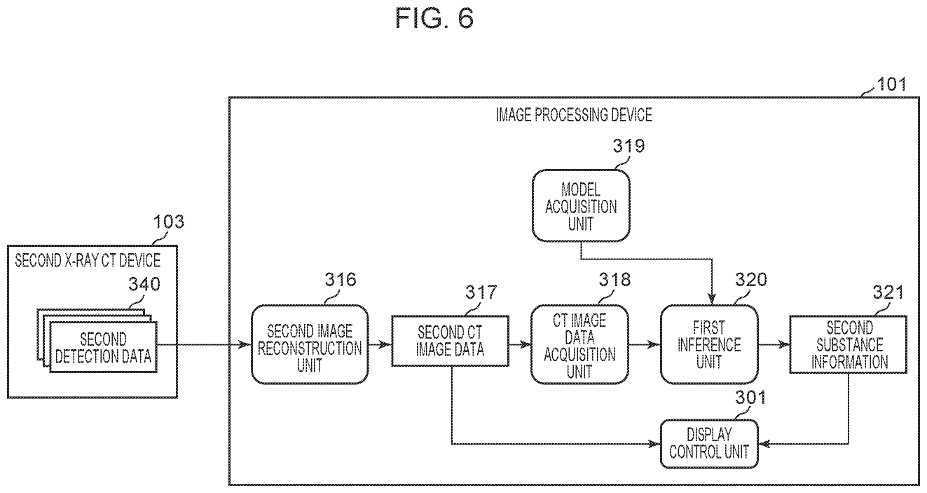

FIG. 3 is a block diagram of an example of the functional configuration of the image processing device 101 according to the present embodiment. The image processing device 101 illustrated in FIG. 3 includes a first image reconstruction unit 310, a first substance information generation unit 312, and a first machine learning unit 314. The image processing device 101 further includes a second image reconstruction unit 316, a CT image data acquisition unit 318, a first inference unit 320 for obtaining second substance information, and a display control unit 301. As described below with reference to, for example, FIGS. 4 and 6, the functional configuration may be achieved as an image processing system including a plurality of image processing devices. The learning function and the inference function may be performed by different image processing devices.

The first image reconstruction unit 310 acquires, from the first X-ray CT device 102, a plurality of first detection data 330 obtained by the first X-ray CT device 102 that captures the images of a plurality of objects. Instead of directly acquiring the first detection data 330 from the first X-ray CT device 102 via the LAN 104, the first image reconstruction unit 310 may acquire the first detection data 330 from a storage unit or the like that stores the first detection data 330.

Subsequently, the first image reconstruction unit 310 reconstructs a first CT image data 311 on the basis of each of the plurality of first detection data 330. At this time, the first detection data 330 are sinograms divided into X-ray energy ranges. A sinogram is data arranged such that the X-axis represents the arrangement of detectors and the Y-axis represents the projection position (the rotation angle). In image reconstruction, the first image reconstruction unit 310 integrates (energy-integrates) the first detection data 330 in the X-ray energy direction to obtain the first CT image data from the energy-integrated sinogram by using an existing back-projection method or successive approximation image reconstruction method. Each of first CT image data 311 is generated from one of the plurality of first detection data 330 by the first image reconstruction unit 310.

The first substance information generation unit 312 obtains the same plurality of first detection data 330 as described above from the first X-ray CT device 102 and generates first substance information 313. The first substance information 313 is information indicating a region of the image corresponding to a predetermined substance and is a masked image having voxel values (pixel values) according to the type of substance. A plurality of pieces of first substance information 313 are generated for each of the plurality of first detection data 330.

In the first substance information generation unit 312, the first substance information 313 is generated by the following procedure. First, the first substance information generation unit 312 reconstructs the first detection data 330 for each of the X-ray energy ranges to obtain monochromatic X-ray image data for the X-ray energy range. Subsequently, the first substance information generation unit 312 evaluates the degree of coincidence between the distribution of the voxel values in the X-ray energy direction obtained from the monochromatic X-ray image data for each of the X-ray energy ranges and the distribution of HU values in the X-ray energy direction assumed from the attenuation coefficient of the substance for the X-ray energy range.

For example, if the X-ray energy ranges are 80 kV and 140 kV, the HU value ratio assumed from the attenuation coefficient is about 1.7 for iodine and is about 1.3 for calcium. In this case, the first substance information generation unit 312 evaluates that if the voxel value ratio in the case of 80 kV and 140 kV monochromatic X-ray images is close to 1.7, the substance has a high degree of coincidence with iodine and, if the voxel value ratio is close to 1.3, the substance has a high degree of coincidence with calcium. Subsequently, the first substance information generation unit 312 identifies the substance having a degree of coincidence that meets a predetermined criterion for each of the voxels in the image. Finally, the first substance information generation unit 312 assigns a numerical value (a label value) preassigned to the identified substance to the position in the masked image corresponding to each voxel in the image. Note that the above-described number of X-ray energy ranges, the HU value ratio between X-ray energy ranges, and the substances to be identified are only examples and are not limited thereto.

The first machine learning unit 314 performs machine learning using a training data set including training data that includes the first CT image data 311 and the first substance information 313 to generate a first trained model 315. The first machine learning unit 314 may perform machine learning using a training data set that includes the plurality of first CT image data 311 and the plurality of pieces of first substance information 313 as training data to generate the first trained model 315. More specifically, the first trained model 315 is an image generation model that receives the first CT image data 311 as an input and outputs the first substance information 313 and is based on a convolutional neural network (CNN), such as U-Net. In machine learning, a variety of parameters of the CNN are updated to reduce an error between the output value obtained by inputting the first CT image data 311 to the CNN and the first substance information 313 (generated from the same first detection data 330) that is paired with the input first CT image data 311, and the update is repeated for a plurality of images. The first trained model 315 may be an image generation model based on an algorithm other than CNN, such as a DNN (Deep Neural Network), vision transformer, or SVM (Support Vector Machine). In image generation models based on an algorithm primarily dealing with a classification task (for example, SVM), the likelihood of a substance is estimated for each of divided regions of an image, and the estimation results are combined to generate a masked image (for example, the sliding window method). The generation of a masked image by the image generation model is also referred to as “segmentation”.

The second image reconstruction unit 316 acquires second detection data 340 from the second X-ray CT device 103 and reconstructs the second detection data 340 into second CT image data 317. The second X-ray CT device 103 is a CT device including an energy-integrating radiation detector that uses a detection method different than that of the first X-ray CT device 102. The second detection data 340 is a sinogram, which is data arranged such that the X-axis represents the arrangement of detectors and the Y-axis represents the projection position (the rotation angle). In image reconstruction performed by the second image reconstruction unit 316, the second CT image data 317 (a plurality of tomographic image data) are obtained by using an existing back-projection method or successive approximation image reconstruction method. Instead of acquiring the second detection data 340 directly from the second X-ray CT device 103 via the LAN 104, the second image reconstruction unit 316 may acquire the second detection data 340 from a storage unit or the like that stores the second detection data 340.

The CT image data acquisition unit 318 acquires the second CT image data 317 based on the second detection data 340 from a storage unit or the like. The second CT image data 317 is image data to be inferred.

The first inference unit 320 that obtains the second substance information causes the first trained model 315, which has been trained by the first machine learning unit 314, to infer second substance information 321 from the second CT image data 317. More specifically, the first inference unit 320 inputs the second CT image data 317 to the first trained model 315 and obtains the output as the second substance information 321.

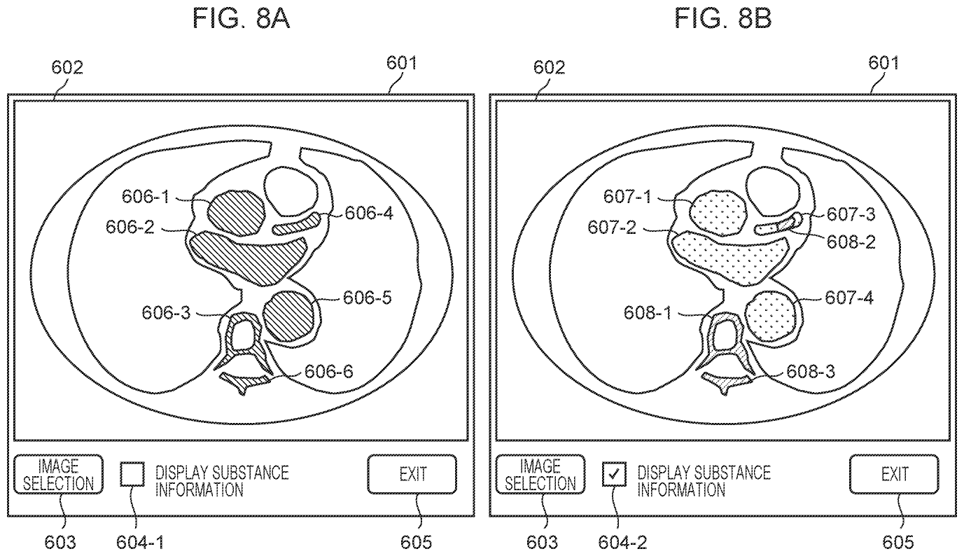

The display control unit 301 displays the second CT image data 317 and the second substance information 321 on the display 207, together with a display screen 601 (described below with reference to FIGS. 8A and 8B).

### Flow of First Learning Process

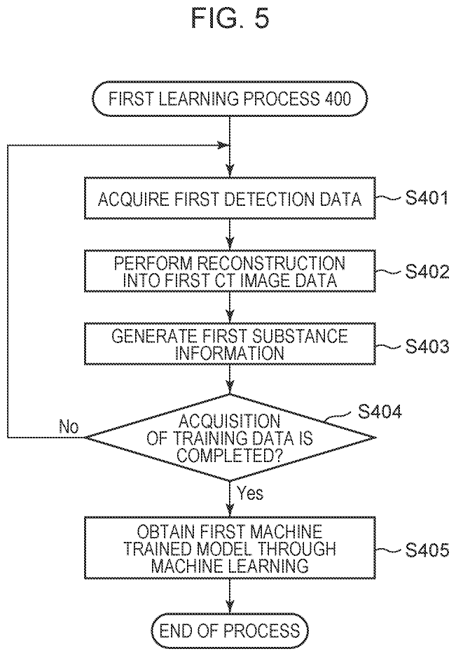

A learning process step of a first inference model according to the present embodiment is described below with reference to FIGS. 4 and 5. FIG. 4 is an example of the functional configuration of the image processing device 101 relating to a learning process. FIG. 5 is a flowchart of an example of a first learning process 400 according to the present embodiment.

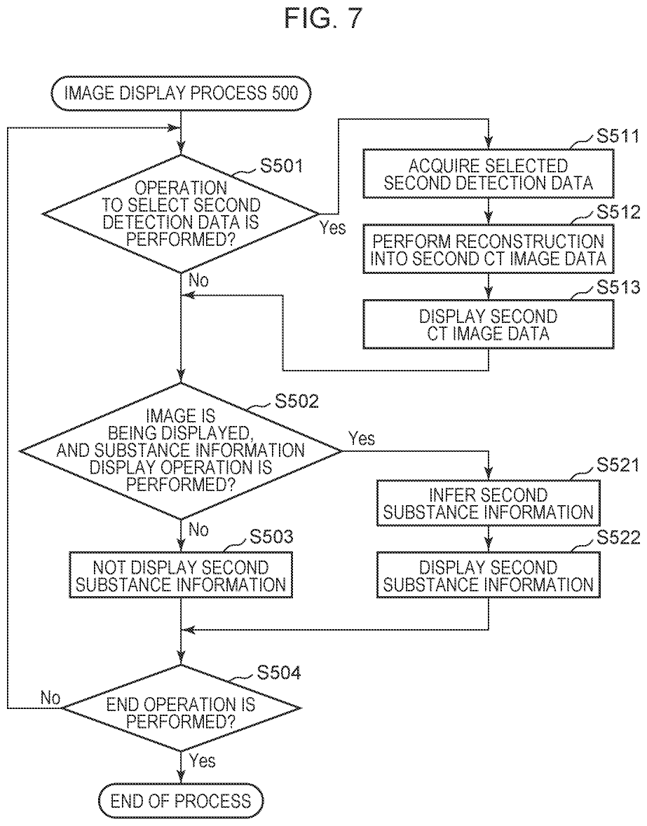

The first learning process 400 illustrated in FIG. 5 is initiated automatically or manually prior to an image display process 500 (described below with reference to FIGS. 6 and 7). The first learning process 400 may be repeated multiple times.

In step S401, the first image reconstruction unit 310 and/or the first substance information generation unit 312 acquires the first detection data 330 acquired by capturing the image of an object using the first X-ray CT device 102 from the first X-ray CT device 102 via the LAN 104. To acquire the first detection data 330 via the LAN 104, an existing Internet protocol, such as HTTP (Hypertext Transfer Protocol, is used. The first detection data 330 may be acquired from a storage device, such as a database, that stores the data captured by the first X-ray CT device 102.

In step S402, the first image reconstruction unit 310 reconstructs the first detection data 330 acquired in step S401 into the first CT image data 311.

In step S403, the first substance information generation unit 312 generates the first substance information 313 from the first detection data 330 acquired in step S401.

In step S404, the first machine learning unit 314 determines whether the acquisition of the training data has been completed. More specifically, the first machine learning unit 314 determines whether the number of the first CT image data 311 reconstructed in step S402 and the number of pieces of the first substance information 313 generated in step S403 have reached a predetermined number. If the acquisition of the training data has not been completed (No in step S404), the processing from step S401 is repeated. If the acquisition of the training data has been completed (Yes in step S404), the processing proceeds to step S405.

In step S405, the first machine learning unit 314 performs a machine learning process using the training data set and generates the first trained model 315. Then, the processing ends. The training data set includes the plurality of first CT image data 311 reconstructed in step S402 and the plurality of pieces of first substance information 313 generated in step S403.

### Flow of Image Display Process

The image display process step according to the present embodiment is described with reference to FIGS. 6 and 7. FIG. 6 illustrates an example of the functional configuration of the image processing device 101 relating to the image display process step.

FIG. 7 is a flowchart of an example of the image display process 500 according to the present embodiment. The image display process 500 is initiated in response to a user operation when a user views a CT image.

In step S501, the second image reconstruction unit 316 determines whether an operation to select the second detection data 340 has been performed on the basis of the operation input to the display screen 601 (described below with reference to FIGS. 8A and 8B) via the keyboard 209 or mouse 210. Hereinafter, the selection target is also simply referred to as an “image”. If the selection operation is performed (Yes in step S501), the processing proceeds to step S511. If the selection operation is not performed (No in step S501), the processing proceeds to step S502.

In step S511, the second image reconstruction unit 316 acquires, from the second X-ray CT device 103, the second detection data 340 selected in step S501 via the LAN 104. To acquire the second detection data 340 via the LAN 104, an existing Internet protocol, such as HTTP (Hypertext Transfer Protocol), is used. Instead of directly acquiring the second detection data 340, the second image reconstruction unit 316 may acquire the second detection data 340 from a storage device, such as a database, that stores data captured by the second X-ray CT device 103.

In step S512, the second image reconstruction unit 316 reconstructs the second detection data 340 acquired in step S511 into the second CT image data 317.

In step S513, the display control unit 301 displays the second CT image data 317 obtained through reconstruction in step S512 on the display 207, together with the display screen 601 (described below with reference to FIG. 6).

In step S502, the first inference unit 320 determines whether the second CT image data 317 is being displayed in step S513 and whether a substance information display operation has been performed. The processing up to step S513 may be performed by another device or agent, and the processing may be initiated from the step in which the CT image data acquisition unit 318 acquires the second CT image data 317.

The presence or absence of the substance information display operation is determined on the basis of the operation input to the display screen 601 (described below with reference to FIGS. 8A and 8B) via the keyboard 209 and mouse 210. If the image is being displayed (that is, the second CT image data 317 is selected) and a substance information display operation is performed (Yes in step S502), the processing proceeds to step S521. If the image is not being displayed (that is, the second CT image data 317 is not selected) or a substance information display operation is not performed (No in step S502), the processing proceeds to step S503.

In step S521, a model acquisition unit 319 acquires the first trained model 315 generated in the first learning process 400 and sends the first trained model 315 to the first inference unit 320 for acquiring the second substance information. The first inference unit 320 infers the second substance information 321 from the second CT image data 317 reconstructed in step S512.

In step S522, the display control unit 301 superimposes the second substance information 321 generated in step S521 on the second CT image data 317 reconstructed in step S512 and displays the image data on the display 207 together with the display screen 601 (described below with reference to FIGS. 8A and 8B).

In step S503, the display control unit 301 does not superimpose the second substance information 321. That is, if the second CT image data 317 is displayed, the display control unit 301 displays only the second CT image data 317 on the display 207, together with the display screen 601 (described below with reference to FIGS. 8A and 8B).

In step S504, a control unit (not illustrated) determines whether an end operation is performed on the basis of an operation input to the display screen 601 (described below with reference to FIGS. 8A and 8B) via the keyboard 209 or mouse 210. If the end operation is performed (Yes in step S504), the processing ends. If an end operation is not performed (No in step S504), the processing is repeated from step S501.

### Display Screen

FIGS. 8A and 8B illustrate examples of the display screen 601 of the image processing device 101 according to the present embodiment. FIG. 8A illustrates an example of the display screen 601 without substance information displayed thereon, and FIG. 8B illustrates an example of the display screen 601 with substance information displayed thereon.

The display screen 601 in FIGS. 8A and 8B is displayed on the display 207 by the display control unit 301. The display screen 601 includes an image displaying region 602, an image selection button 603, a substance information display check box 604-1 or 604-2, and an exit button 605.

The image selection button 603 is a button for selecting an image. When the image selection button 603 is clicked with the mouse 210, the display control unit 301 displays a screen (not illustrated) for selecting an image to be displayed on the display 207, and the user can select an image to be displayed via the screen. When the image to be displayed is selected by the user, the second image reconstruction unit 316 reconstructs the second detection data 340 corresponding to the image to be displayed into the second CT image data 317, and the display control unit 301 displays the second CT image data 317 in the image displaying region 602. As described above, if the CT image data acquisition unit 318 acquires the second CT image data pre-reconstructed from the second detection data 340, the above-described step may be removed as appropriate.

The image displaying region 602 is a region in which an image is displayed, and the display control unit 301 displays, for example, the cross section of the second CT image data 317. Regions 606-1 to 606-6 are examples of regions having voxel values (HU values) in a predetermined range in the displayed cross section.

The substance information display check box 604-1 is a check box to specify whether the substance information is displayed, and the substance information display check box 604-1 is an example of the check box that is unchecked. When the substance information display check box 604-1 is unchecked, the display control unit 301 does not display the second substance information 321 in the image displaying region 602. Therefore, the regions 606-1 to 606-6 having voxel values (HU values) in the predetermined range are displayed so that substances are not discriminated.

The substance information display check box 604-2 is an example of the check box that is checked to display the substance information, and the display control unit 301 displays the second substance information 321 superimposed on the second CT image data 317 in the image displaying region 602.

Regions 607-1 to 607-4 and regions 608-1 to 608-3 are examples of displayed regions in which substances are discriminated. The regions 607-1 to 607-4 are regions identified as iodine, and the regions 608-1 to 608-3 are regions identified as calcium.

The exit button 605 is used to select the end of the process. When the exit button 605 is clicked with the mouse 210, a control unit (not illustrated) terminates the image display process 500 and display of the display screen 601.

### Modification of First Embodiment

The image processing device 101 according to the first embodiment may be an image processing workstation, a console of an X-ray CT device, or an image processing server.

The image processing device 101 according to the first embodiment may be configured not to select the second detection data 340 based on a user operation or display the second substance information 321 by the display control unit 301. In this case, the first image reconstruction unit 310 and/or the first substance information generation unit 312 may acquire (receive) the second detection data 340 output from an external device (for example, a server or a viewer (not illustrated)) in step S501. Alternatively, the first image reconstruction unit 310 may receive designation of the second detection data 340 from an external device in step S501 and acquire the designated second detection data 340 from an external device in step S511. When the first image reconstruction unit 310 acquires (receives) the second detection data 340 in step S501, the process in step S511 is not necessary. Immediately after the process in step S513 is performed, the process in step S521 is performed. In step S521, the first inference unit 320 outputs the second substance information 321 to an external device, such as a server or a viewer (not illustrated). In this case, the processes in steps S502, S503, and S522 are not necessary.

The first detection data 330 according to the first embodiment may be stored in a device other than the first X-ray CT device 102 (for example, a server provided separately (not illustrated)) and be acquired by the first image reconstruction unit 310 and/or the first substance information generation unit 312 of the image processing device 101. In this case, in step S401, the first image reconstruction unit 310 and/or the first substance information generation unit 312 acquires the first detection data 330 from the device.

The first image reconstruction unit 310 according to the first embodiment may be provided in a device other than the image processing device 101 (for example, the first X-ray CT device 102). In this case, the first machine learning unit 314 of the image processing device 101 may be configured to acquire the first CT image data 311 from the device. Alternatively, the first machine learning unit 314 of the image processing device 101 may be configured to acquire the first CT image data 311 stored in a separately provided server (not illustrated). In this case, in step S402 according to the first embodiment, instead of the process in which the first image reconstruction unit 310 acquires the first CT image data 311 through reconstruction, the first machine learning unit 314 acquires the first CT image data 311 stored outside.

Similarly, the first substance information generation unit 312 according to the first embodiment may be provided in a device other than the image processing device 101 (for example, the first X-ray CT device 102). In this case, configuration may be such that the first machine learning unit 314 of the image processing device 101 acquires the first substance information 313 from the device. Alternatively, the configuration may be such that the first machine learning unit 314 of the image processing device 101 acquires the first substance information 313 stored in a separately provided server (not illustrated). In this case, in step S403 according to the first embodiment, instead of the process in which the first substance information generation unit 312 generates the first substance information 313, the first machine learning unit 314 may acquire the first substance information 313 stored outside.

In the case of a configuration in which both the first CT image data 311 and the first substance information 313 are acquired from outside the image processing device 101, the first detection data 330 is not necessary and, thus, the process in step S401 can be omitted.

The first machine learning unit 314 according to the first embodiment may be provided in a device (not illustrated) other than the image processing device 101, and the first trained model 315 may be stored in a separately provided server (not illustrated) and be acquired by the model acquisition unit 319 of the image processing device 101. In this case, the processes in steps S401 to S404 according to the first embodiment can be omitted. In addition, in step S405, instead of the process in which the first machine learning unit 314 generates the first trained model 315, the second substance information generating unit 318 acquires the first trained model 315 stored in the server (not illustrated).

The second detection data 340 according to the first embodiment may be stored in a separately provided server (not illustrated) and be acquired by the second image reconstruction unit 316 of the image processing device 101. In this case, in step S511, the second image reconstruction unit 316 acquires the second detection data 340 stored in the server (not illustrated).

The second image reconstruction unit 316 according to the first embodiment may be provided in a device other than the image processing device 101 (for example, the second X-ray CT device 103). In this case, the configuration may be such that the CT image data acquisition unit 318 of the image processing device 101 acquires the second CT image data 317 from the device. Alternatively, the configuration may be such that the CT image data acquisition unit 318 acquires the second CT image data 317 stored in a separately provided server (not illustrated). In this case, in step S512 according to the first embodiment, instead of the process in which the second image reconstruction unit 316 acquires the second CT image data 317 through reconstruction, the CT image data acquisition unit 318 acquires the second CT image data 317 stored outside.

The first substance information 313 and the second substance information 321 according to the first embodiment are substance discrimination information. The substance discrimination information may be label information indicating the presence or absence of a predetermined substance for the entire image. Alternatively, the substance discrimination information may be a bounding box indicating the presence of the substance and label information or may be information other than a masked image, such as annotation information (for example, an arrow) and label information.

The first X-ray CT device 102 may be a dual-energy X-ray CT device.

The X-ray CT device may include both a photon counting detector and an integrating detector. In this case, the first image reconstruction unit 310 reconstructs the detection data acquired from the integrating detector into the first CT image data 311, and the first substance information generation unit 312 generates the first substance information 313 from the detection data acquired from the photon counting detector.

That is, the first X-ray CT device 102 is an X-ray CT device capable of acquiring the first CT image data 311 based on the first detection data 330 and the first substance information 313 based on the first detection data. In contrast, the second X-ray CT device 103 is an X-ray CT device incapable of acquiring the substance information.

The first machine learning unit 314 according to the first embodiment may further perform machine learning using data additionally including a pair consisting of image data acquired by any unit and the substance information and may generate the first trained model 315.

The first machine learning unit 314 according to the first embodiment may perform additional learning on a model generated through machine learning using the above-described training data set and generate the first trained model 315.

The first machine learning unit 314 according to the first embodiment may perform additional learning using the above-described training data set on a model generated through machine learning using any training data and may generate the first trained model 315.

The first machine learning unit 314 according to the first embodiment may perform additional learning using the first CT image data 311 and the first substance information 313 as the training data on a model acquired by any unit and may generate the first trained model 315.

The first image reconstruction unit 310 and the first substance information generation unit 312 according to the first embodiment may use, as the training data, data that is a pair consisting of the first CT image data 311 and the first substance information 313 for which the first detection data 330 is the same. The first CT image data 311 and the first substance information 313 that constitute the training data may be output to an external device, such as a server (not illustrated).

The first substance information 313 and the second substance information 321 according to the first embodiment may be a plurality of or multiple-channel masked images each provided for one of substances to be discriminated. In addition, the first trained model 315 may be provided in a plurality, each for one of the substances to be discriminated.

Each of the voxel values (pixel values) of the first substance information 313 according to the first embodiment need not be a binary value indicating whether the degree of coincidence with the distribution of the HU values in the X-ray energy direction for each of the substances to be discriminated is high or low and may be a continuous value that indicates the degree of coincidence as the likelihood of the substance. The substance information for discriminating a substance using the likelihood may be used as the voxel value (pixel value).

The first machine learning unit 314 may perform machine learning using a training data set including, as the training data, intermediate data, such as a monochromatic X-ray image, used to generate the first substance information 313 and the first CT image data 311 and may generate the first trained model 315. In this case, the second substance information generation unit 318 generates the second substance information 321 using the intermediate data, such as the monochromatic X-ray image, generated by the first trained model 315.

As described above, according to the present embodiment and the modification of the present embodiment, a machine-trained model can be constructed using data generated from PCCT or DECT as training data. By applying the trained model, highly accurate substance information can be obtained from even CT image data acquired from an existing X-ray CT device including an energy-integrating X-ray detector.

### Second Embodiment

In addition to the processing according to the first embodiment, an image processing device according to the present embodiment infers the second CT image data from the first detection data using a second trained model that has been trained using a training data set that includes the first detection data and the second CT image data as the training data. The hardware configurations of an image processing system 100 and an image processing device 101, a first learning process 400, an image display process 500, and a display screen 601 according to the present embodiment are the same as those according to the first embodiment and, therefore, their descriptions are omitted.

### Functional Configuration

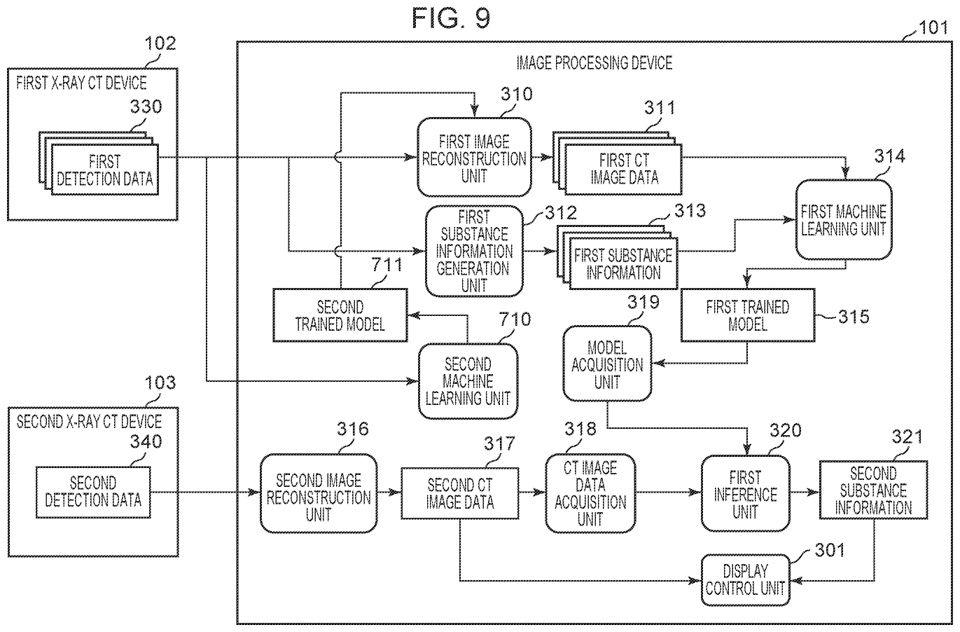

FIG. 9 is a block diagram of an example of the functional configuration of the image processing device 101 according to the present embodiment. A configuration described in another embodiment is identified with the same reference numeral, and description of the configuration is omitted as appropriate. The image processing device 101 described in FIG. 9 has the configuration of the image processing device 101 according to the first embodiment described in FIG. 3 and further includes a second machine learning unit 710.

The second machine learning unit 710 performs machine learning using a training data set and generates a second trained model 711. The training data set includes a plurality of first detection data 330 obtained by capturing the images of a plurality of objects and a plurality of second CT image data 317 each corresponding to one of the plurality of first detection data 330. The first detection data 330 and the second CT image data 317 are data acquired by capturing the image of the same object with the first X-ray CT device 102 and the second X-ray CT device 103, respectively. The second machine learning unit 710 performs an alignment process so that the second CT image data 317 used as the training data has the same object position in the image as the first CT image data 311 obtained by reconstructing the first detection data using the first image reconstruction unit 310. Note that the first image reconstruction unit 310 reconstructs the first CT image data 311 used for the alignment process using the energy integration and the back-projection method or the successive approximation image reconstruction method described in the first embodiment. The second trained model 711 generated by the second machine learning unit 710 is an image generation model that receives the first detection data 330 as an input and outputs the second CT image data 317 and is based on a CNN (convolutional neural network), such as U-Net.

In the learning process performed by the second machine learning unit 710, a variety of parameters of the CNN are updated so that an error is reduced between the output value obtained by inputting the first detection data 330 into the CNN and the second CT image data 317 (generated from the second detection data 340 acquired by capturing the image of the same object as the first detection data 330) that is paired with the input first detection data 330 and is subjected to the above-described alignment process. The updating of the CNN parameters is repeated for the plurality of images. The second trained model 711 may be an image generation model based on an algorithm other than a CNN, such as a deep neural network (DNN) or vision transformer.

The first image reconstruction unit 310 may use the second trained model 711 that is machine-learned in the second machine learning unit 710 to reconstruct the first detection data 330 into the first CT image data 311.

### Flow of Second Learning Process

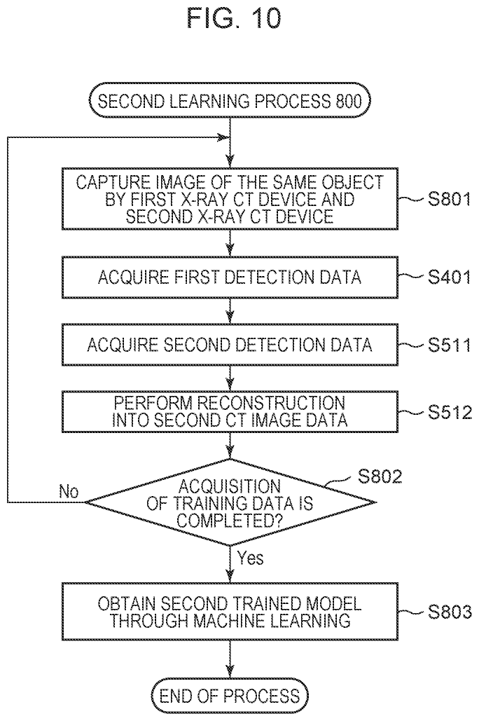

FIG. 10 is a flowchart of an example of a second learning process 800 according to the second embodiment. The second learning process 800 is initiated automatically or manually prior to the first learning process 400 (described with reference to FIG. 4). The second learning process 800 may be repeated multiple times.

In step S801, control units (not illustrated) of the first X-ray CT device 102 and the second X-ray CT device 103 capture the images of the same object on the basis of a user operation. The image capturing may be performed at different times. If one of the two data is present, only the other data that is not present may be acquired. If both data are present, step S801 may be skipped as appropriate. Instead of capturing the image, the captured data may be retrieved from a storage unit in step S801.

In step S802, the second machine learning unit 710 determines whether acquisition of the training data has been completed. More specifically, the second machine learning unit 710 determines whether the number of training data, which is a pair consisting of the first detection data 330 acquired in step S401 and the second CT image data 317 obtained through reconstruction in step S512, has reached a preset number. If the acquisition of the training data has not been completed (No in step S802), the processes in step S801 and the subsequent steps are repeated. If the acquisition of the training data has been completed (Yes in step S802), the processing proceeds to step S803.

In step S803, the second machine learning unit 710 performs a process of aligning the second CT image data 317 with the first CT image data 311 obtained by reconstructing the first detection data 330. The second machine learning unit 710 performs machine learning using a training data set including, as training data, the plurality of first detection data 330 and the plurality of aligned second CT image data 317 and generates the second trained model 711 and, then, the processing ends.

### Modification of Second Embodiment

The second machine learning unit 710 according to the second embodiment may be provided in a device (not illustrated) other than the image processing device 101, and the second trained model 711 may be stored in a separately provided server (not illustrated) and be acquired by the first image reconstruction unit 310 of the image processing device 101. In this case, the processes in steps S801 to S802 of the second embodiment can be omitted. In addition, in step S803, instead of the process performed by the second machine learning unit 710 to generate the second trained model 711, the first image reconstruction unit 310 acquires the second trained model 711 stored in a server (not illustrated).

The second trained model 711 according to the second embodiment may be an image generation model that receives the first CT image data obtained by reconstructing the first detection data 330 by energy integration and back-projection method or successive approximation image reconstruction method as an input and outputs the second CT image data 317. In this case, the second machine learning unit 710 performs machine learning using a training data set including training data that receives the reconstructed first CT image data as an input and outputs the second CT image data 317 to generate the second trained model 711. The first image reconstruction unit 310 obtains the first CT image data by reconstructing the first detection data 330 using energy integration and a back-projection method or successive approximation image reconstruction method. In addition, the first image reconstruction unit 310 may acquire the second CT image data, which is the output obtained by inputting the first CT image data into the second trained model 711. Thereafter, the first trained model described above may be generated using a training data set that includes, as training data, the second CT image data and the first substance information that form a pair.

The first detection data 330 and the second detection data 340 may be generated so that the objects in the images are aligned. More specifically, a detector for acquiring the first detection data 330 and a detector for acquiring the second detection data 340 are installed in the same movable portion of the X-ray CT device, and image capturing is performed. In this case, the image alignment process between the second CT image data 317 and the first CT image data 311 performed in the second machine learning unit 710 in step S803 is not necessary.

The second machine learning unit 710 according to the second embodiment may generate the second trained model 711 by using training data that includes data to which a pair consisting of detection data and image data acquired by any other unit is added.

The second machine learning unit 710 according to the second embodiment may acquire a model generated through machine learning using a training data set that includes, as training data, the first detection data 330 and the second CT image data 317. Furthermore, the second machine learning unit 710 may perform additional learning on the model using a training data set including, as training data, a pair consisting of detection data and image data acquired by any unit and generate the second trained model 711.

The second machine learning unit 710 according to the second embodiment may perform additional learning using the above-described training data set on a model generated through machine learning using a training data set acquired by any unit and generate the second trained model 711.

The second machine learning unit 710 according to the second embodiment may perform additional learning using the first detection data 330 and the second CT image data 317 as training data on a model acquired by any unit and generate the second trained model 711.

As described above, according to the present embodiment and the modification of the present embodiment, a machine-trained model can be constructed using data generated from PCCT or DECT as training data. By applying the trained model, highly accurate substance information can be obtained from even CT image data acquired by an existing X-ray CT device including an energy-integrating X-ray detector.

Furthermore, by using the second trained model 711, the acquired first CT image data 311 becomes similar to the second CT image data 317 (that is, the image captured by an energy-integrating X-ray CT device), so that the first trained model 315 is optimized for the second CT image data 317, enabling substance discrimination with improved performance.

### Third Embodiment

Unlike the first embodiment, an image processing device 101 according to the present embodiment obtains a third trained model using a training data set that includes, as training data, a first sinogram based on the first detection data and the first substance information. In addition, the image processing device 101 infers the third substance information from the second sinogram based on the second detection data by using the third trained model subjected to learning processing. Since the hardware configurations of the image processing system 100 and image processing device 101 and the display screen 601 according to the present embodiment are the same as those according to the first embodiment, their description is omitted. In addition, since a first learning process 400 and an image display process 500 are similar to those according to the first embodiment by replacing the image data with a sinogram and image reconstruction with generation of sinograms, their description is omitted.

### Functional Configuration

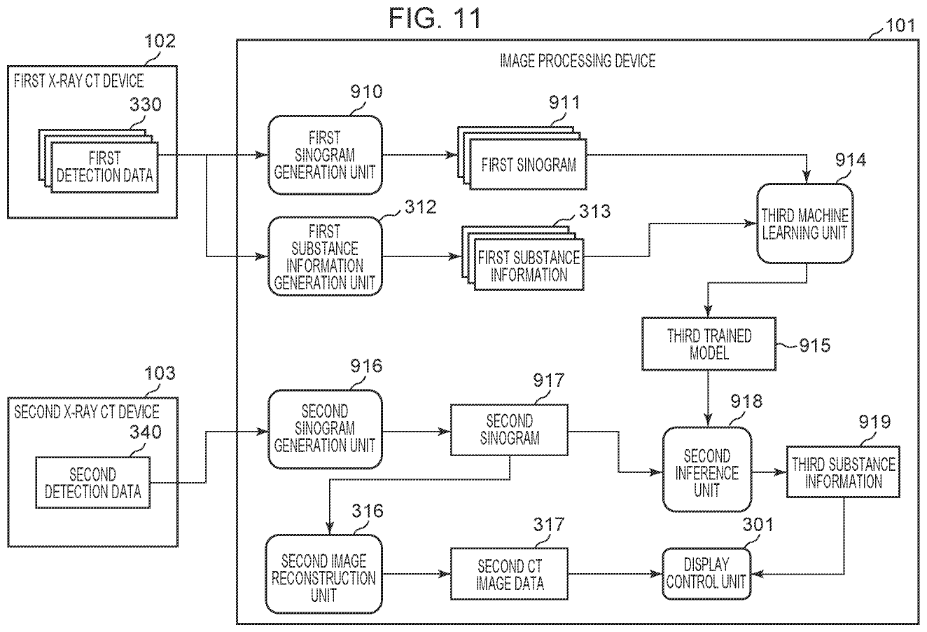

FIG. 11 is a block diagram of an example of the functional configuration of the image processing device 101 according to the present embodiment. Configurations described in other embodiments are identified by the same reference numerals, and their description is omitted. In the image processing device 101 illustrated in FIG. 11, a first sinogram generation unit 910 is substituted for the first image reconstruction unit 310 of the image processing device 101 according to the first embodiment illustrated in FIG. 3, a third machine learning unit 914 is substituted for the first machine learning unit 314, and a second inference unit 918 is substituted for the first inference unit 320. In addition, a second sinogram generation unit 916 is additionally provided.

The first sinogram generation unit 910 acquires the first detection data 330 from the first X-ray CT device 102 and generates a first sinogram 911. The first detection data 330 is a sinogram divided into X-ray energy ranges, and the first sinogram 911 is an energy-integrated sinogram. Therefore, the first sinogram generation unit 910 generates the first sinogram by integrating (energy-integrating) the first detection data 330 in the X-ray energy direction.

The third machine learning unit 914 performs machine learning using the plurality of first sinograms 911 and the plurality of pieces of first substance information 313 as training data and generate a third trained model 915. More specifically, the third trained model 915 is an image generation model that receives the first sinogram 911 as an input and outputs the first substance information 313 and is based on a CNN (convolutional neural network), such as U-Net.

In the machine learning, a variety of parameters of the CNN are updated to reduce an error between the output value obtained by inputting the first sinogram 911 and the first substance information 313 (generated from the same first detection data 330) that is paired with the input first sinogram 911, and the update is repeated for a plurality of images. The third trained model 915 may be an image generation model based on an algorithm other than CNN, such as a DNN (Deep Neural Network), vision transformer, or SVM (Support Vector Machine). In image generation models based on an algorithm primarily dealing with a classification task (for example, SVM), the likelihood of a substance is estimated for each of divided regions of an image, and the estimation results are combined to generate a masked image (for example, the sliding window method). The generation of a masked image by the image generation model is also referred to as “segmentation”.

The second sinogram generation unit 916 generates, from the second detection data 340, a second sinogram 917 which is data arranged such that the X-axis represents the arrangement of detectors and the Y-axis represents the projection position (the rotation angle). If the second detection data 340 is a sinogram acquired from an energy-integrating detector, the second sinogram 917 is the same as the second detection data 340.

The second inference unit 918 uses the third trained model 915 to infer third substance information 919 corresponding to the second sinogram 917. More specifically, the output obtained by inputting the second sinogram 917 into the third trained model 915 is defined as the third substance information 919.

The display control unit 301 displays the second CT image data 317 and the third substance information 919 on the display 207, together with the display screen 601 (described above with reference to FIG. 6).

### Modification of Third Embodiment

Like the modification of the first embodiment, the first sinogram generation unit 910, the first substance information generation unit 312, the third machine learning unit 914, and the second sinogram generation unit 916 of the image processing device 101 according to the third embodiment may be provided in a device other than the image processing device 101.

The first sinogram generation unit 910 of the image processing device 101 according to the third embodiment may perform machine learning using a training data set that includes a plurality of first detection data 330 and a plurality of second sinograms 917 as training data. The first sinogram generation unit 910 may generate the first sinogram 911 using a generated trained model. More specifically, the trained model is an image generation model that receives the first detection data 330 as an input and outputs the second sinogram 917 and is based on a convolutional neural network (CNN), such as U-Net. In machine learning, a variety of parameters of the CNN are updated to reduce an error between the output value obtained by inputting the first detection data 330 and the second sinogram 917 (generated from the second detection data 340 acquired by capturing the same object as the first detection data 330) that is paired with the input first detection data 330, and the update is repeated for a plurality of images. The trained model may be an image generation model based on an algorithm other than CNN, such as a DNN (Deep Neural Network) or a vision transformer.

As described above, according to the present embodiment and the modification of the present embodiment, a machine-trained model can be constructed using data generated from PCCT or DECT as training data. By applying the trained model, highly accurate substance information can be obtained from even CT image data acquired from an existing X-ray CT device including an energy-integrating X-ray detector.

Furthermore, by using a sinogram before reconstruction, a trained model that is not influenced by the reconstruction function or the like is provided, and substance information can be obtained with reduced influence of a difference between the reconstruction functions.

### Other Embodiments

The present disclosure is also realized by performing the following processes. That is, the software (program) that realizes the functions of the above-described embodiments is supplied to a system or a device via a network or a variety of storage media, and the computer (or a CPU, an MPU, or the like) of the system or device reads and executes the program.

The disclosure according to the embodiments of the present disclosure includes the following configurations and methods.

### Configuration 1

An image processing device includes a model acquisition unit configured to acquire a first trained model trained using a training data set including, as training data, first CT image data based on first detection data that is captured by a first X-ray CT device and first substance information based on the first detection data, a CT image data acquisition unit configured to acquire second CT image data based on second detection data that is captured by a second X-ray CT device including an energy-integrating radiation detector using a detection method different from a detection method used by the first X-ray CT device, and an inference unit configured to infer second substance information from the second CT image data by using the first trained model.

### Configuration 2

In the image processing device according to Configuration 1, the above-described first X-ray CT device is one of a dual-energy CT device and a photon counting CT device.

### Configuration 3

The image processing device according to Configuration 1 or 2 further includes a first substance information acquisition unit configured to acquire the first substance information from the first detection data.

### Configuration 4

In the image processing device according to any one of Configurations 1 to 3, the first X-ray CT device is an X-ray CT device capable of acquiring substance information based on the first CT image data based on the first detection data and the first detection data, and the second X-ray CT device is an X-ray CT device capable of acquiring substance information based on the second detection data.

### Configuration 5

In the image processing device according to any one of Configurations 1 to 4, the first substance information is acquired based on pixel values that constitute a plurality of image data each corresponding to one of a plurality of energy ranges reconstructed from the first detection data.

### Configuration 6

In the image processing device according to any one of Configurations 1 to 5, each of the first substance information and the second substance information is information indicating a region in an image corresponding to a predetermined substance.

### Configuration 7

In the image processing device according to any one of Configurations 1 to 6, each of the first substance information and the second substance information is information indicating whether a predetermined substance is present in an image.

### Configuration 8

In the image processing device according to any one of Configurations 1 to 6, each of the first substance information and the second substance information is information indicating a likelihood of a predetermined region or predetermined pixels in an image being a predetermined substance.

### Configuration 9

In the image processing device according to any one of Configurations 1 to 8, the first CT image data that constitutes the training data set includes image data generated from the first detection data by using a second trained model trained using a training data set including the first detection data and the second CT image data as training data.

### Configuration 10

In the image processing device according to any one of Configurations 1 to 9, the first CT image data that constitutes the training data set includes image data generated from the first CT image data by using a second trained model trained using a training data set including the first CT image data and the second CT image data as training data.

### Configuration 11

In the image processing device according to Configuration 9 or 10, the second CT image data that constitutes the training data is image data that is aligned with the first CT image data generated from the first detection data so that positions of an object are the same in images.

### Configuration 12

In the image processing device according to Configuration 11, the second CT image data that constitutes the training data is image data generated from the second detection data captured so that a position of a captured object in an image is the same as a position of the object in the first CT image data.

### Configuration 13

In the image processing device according to any one of Configurations 1 to 12, the first trained model is a trained model that receives CT image data as an input and outputs substance information, and the inference unit infers the second substance information by inputting the second CT image data to the first trained model.

### Configuration 14

In the image processing device according to Configuration 9, the second trained model is a trained model that receives first detection data as an input and outputs second CT image data, and the first CT image data that constitutes the training data includes CT image data generated by inputting the first detection data to the second trained model.

### Configuration 15

An image processing device includes an inference unit configured to use a third trained model trained using, as training data, data including a first sinogram based on first detection data captured by a first X-ray CT device and first substance information and infer third substance information from a second sinogram based on second detection data captured by a second X-ray CT device using a detection method different from a detection method used by the first X-ray CT device.

### Configuration 16

An image processing method includes acquiring a first trained model trained using a training data set including, as training data, first CT image data based on first detection data that is captured by a first X-ray CT device and first substance information based on the first detection data, acquiring second CT image data based on second detection data that is captured by a second X-ray CT device including an energy-integrating radiation detector using a detection method different from a detection method used by the first X-ray CT device, and inferring second substance information from the second CT image data by using the first trained model.

### Configuration 17

An image processing method includes inferring, by using a third trained model trained using, as training data, data including a first sinogram based on first detection data captured by a first X-ray CT device and first substance information, third substance information from a second sinogram based on second detection data captured by a second X-ray CT device configured to use a detection method different from a detection method used by the first X-ray CT device.

### Configuration 18

A non-transitory computer-readable storage medium stores one or more programs including executable instructions, which when executed by a computer, cause the computer to perform the image processing method according to Configuration 16 or 17.

### Configuration 19

An image processing system includes a model acquisition unit configured to acquire a first trained model trained using a training data set including, as training data, first CT image data based on first detection data that is captured by a first X-ray CT device and first substance information based on the first detection data, a CT image data acquisition unit configured to acquire second CT image data based on second detection data that is captured by a second X-ray CT device including an energy-integrating radiation detector using a detection method different from a detection method used by the first X-ray CT device, and an inference unit configured to infer second substance information from the second CT image data by using the first trained model.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2023-200148 filed Nov. 27, 2023, which is hereby incorporated by reference herein in its entirety.

What is claimed is:

1. An image processing device comprising:
at least one memory storing instructions; and
at least one processor that, upon execution of the instructions, configures the at least one processor to:
acquire a first trained model trained using a training data set including, as training data, first CT image data based on first detection data that is captured by a first X-ray CT device and first substance information based on the first detection data;
acquire second CT image data based on second detection data that is captured by a second X-ray CT device including an energy-integrating radiation detector using a detection method different from a detection method used by the first X-ray CT device; and
infer second substance information from the second CT image data by using the first trained model.

2. The image processing device according to claim 1, wherein the first X-ray CT device is one of a dual-energy CT device and a photon counting CT device.

3. The image processing device according to claim 1, further comprising:
a first substance information acquisition unit configured to acquire the first substance information from the first detection data.

4. The image processing device according to claim 1, wherein the first X-ray CT device is an X-ray CT device that acquires substance information based on the first CT image data based on the first detection data and the first detection data, and
wherein the second X-ray CT device is an X-ray CT device that is not able to acquire substance information based on the second detection data.

5. The image processing device according to claim 1, wherein the first substance information is acquired based on pixel values that constitute a plurality of image data each corresponding to one of a plurality of energy ranges reconstructed from the first detection data.

6. The image processing device according to claim 1, wherein each of the first substance information and the second substance information is information indicating a region in an image corresponding to a predetermined substance.

7. The image processing device according to claim 1, wherein each of the first substance information and the second substance information is information indicating whether a predetermined substance is present in an image.

8. The image processing device according to claim 1, wherein each of the first substance information and the second substance information is information indicating a likelihood of a predetermined region or predetermined pixels in an image being a predetermined substance.

9. The image processing device according to claim 1, wherein the first CT image data that constitutes the training data set includes image data generated from the first detection data by using a second trained model trained using a training data set including the first detection data and the second CT image data as training data.

10. The image processing device according to claim 1, wherein the first CT image data that constitutes the training data set includes image data generated from the first CT image data by using a second trained model trained using a training data set including the first CT image data and the second CT image data as training data.

11. The image processing device according to claim 9, wherein the second CT image data that constitutes the training data is image data that is aligned with the first CT image data generated from the first detection data so that positions of an object are the same in images.

12. The image processing device according to claim 11, wherein the second CT image data that constitutes the training data is image data generated from the second detection data captured so that a position of a captured object in an image is the same as a position of the object in the first CT image data.

13. The image processing device according to claim 1, wherein the first trained model is a trained model that receives CT image data as an input and outputs substance information, and
wherein the inference unit infers the second substance information by inputting the second CT image data to the first trained model.

14. The image processing device according to claim 9, wherein the second trained model is a trained model that receives first detection data as an input and outputs second CT image data, and
wherein the first CT image data that constitutes the training data includes CT image data generated by inputting the first detection data to the second trained model.

15. An image processing device comprising:
an inference unit configured to use a trained model trained using, as training data, data including a first sinogram based on first detection data captured by a first X-ray CT device and first substance information and
infer substance information from a second sinogram based on second detection data captured by a second X-ray CT device using a detection method different from a detection method used by the first X-ray CT device.

16. An image processing method comprising:
acquiring a first trained model trained using a training data set including, as training data, first CT image data based on first detection data that is captured by a first X-ray CT device and first substance information based on the first detection data;
acquiring second CT image data based on second detection data that is captured by a second X-ray CT device including an energy-integrating radiation detector using a detection method different from a detection method used by the first X-ray CT device; and
inferring second substance information from the second CT image data by using the first trained model.

17. An image processing method comprising:
inferring, by using a trained model trained using, as training data, data including a first sinogram based on first detection data captured by a first X-ray CT device and first substance information, substance information from a second sinogram based on second detection data captured by a second X-ray CT device configured to use a detection method different from a detection method used by the first X-ray CT device.

18. A non-transitory computer-readable storage medium storing one or more programs including executable instructions, which when executed by a computer, cause the computer to perform the method according to claim 16.

19. A non-transitory computer-readable storage medium storing one or more programs including executable instructions, which when executed by a computer, cause the computer to perform the method according to claim 17.

20. An image processing system comprising:
at least one memory storing instructions and at least one processor that, upon execution of the instructions, configures the at least one processor to:
acquire a first trained model trained using a training data set including, as training data, first CT image data based on first detection data that is captured by a first X-ray CT device and first substance information based on the first detection data;
acquire second CT image data based on second detection data that is captured by a second X-ray CT device including an energy-integrating radiation detector using a detection method different from a detection method used by the first X-ray CT device; and
infer second substance information from the second CT image data by using the first trained model.
